(19) United States
(12) Patent Application Publication
(10) Pub. No.: US 2025/0336474 A1
(43) Pub. Date: Oct. 30, 2025

(54) METHOD FOR IDENTIFYING A PARTICULAR GENE

(71) Applicants: Minal Borkar Tripathi, Calgary (CA); Sidharth Singh Bhinder, Calgary (CA); Anmol Singh kapoor, Calgary (CA); Amrita Kaur, Calgary (CA)

(72) Inventors: Minal Borkar Tripathi, Calgary (CA); Sidharth Singh Bhinder, Calgary (CA); Anmol Singh kapoor, Calgary (CA); Amrita Kaur, Calgary (CA)

(21) Appl. No.: 18/646,879

(22) Filed: Apr. 26, 2024

(51) Int. Cl. G16B 30/00; G16B 45/00

(52) U.S. Cl. CPC G16B 30/00; G16B 45/00

(57) ABSTRACT

The present disclosure relates to a method for identifying a particular gene from a tissue sample is disclosed. The method includes obtaining a first sequence from the tissue sample, wherein the sequence is associated with a particular gene. The method further includes comparing the first sequence with a second sequence, wherein the second sequence is associated with another gene. The method further includes determining whether the first sequence is different from the second sequence, and thereby identifying the particular gene.

### CROSS-REFERENCE TO RELATED APPLICATIONS

This application claims the benefit of U.S. Provisional Application No. 63/498,597 titled “A METHOD FOR IDENTIFYING A PARTICULAR GENE” filed by the applicant on Apr. 27, 2023, which is incorporated herein by reference in its entirety.

### FIELD OF THE INVENTION

Embodiments of the present invention relate to the field of genomics and more particularly, relate to a method for identifying a particular gene.

### DESCRIPTION OF THE RELATED ART

Genomics is the study of the structure, function, and evolution of genomes. It is a field at the intersection of genetics and molecular biology, focuses on the structure, function, evolution, mapping, and editing of genomes, the complete set of an organism's deoxyribonucleic acid (DNA), including all of its genes. It involves the sequencing and analysis of deoxyribonucleic acid (DNA) and ribonucleic acid (RNA) molecules, as well as the identification of the genes contained in these molecules. The understanding of the genetic code and the ability to identify and analyze particular genes are essential for the development of new treatments for diseases.

The genes hold the blueprint for an organism's development, functioning, and inheritance, making genomics a crucial discipline in understanding life at its most fundamental level. Since its inception, the primary goal of genomics is decoding the genetic code underlying various traits and diseases. By identifying genes associated with specific traits or conditions, researchers can gain insights into their molecular mechanisms and develop targeted interventions.

This approach has led to breakthroughs in medical genetics, allowing for personalized medicine tailored to an individual's genetic makeup. For example, genomic profiling can guide treatment decisions in cancer by identifying mutations that drive tumor growth and predicting response to therapies.

In addition to this, genomics also plays a crucial role in understanding evolutionary processes. By comparing the genomes of different species, researchers can trace their evolutionary relationships and uncover the genetic changes that have shaped biodiversity over millions of years. This comparative genomics approach has provided insights into the origins of species, the mechanisms of adaptation, and the genetic basis of evolutionary innovations.

Moreover, genomics has revolutionized agriculture and food security. Through genome sequencing and molecular breeding techniques, scientists can develop crops with improved traits such as yield, disease resistance, and nutritional content. This genomic revolution in agriculture offers promising solutions to global challenges such as climate change, population growth, and sustainable food production.

Identification of a particular gene is of paramount importance in genomics. Fundamentally, identifying a gene allows study of its function within an organism. By understanding the roles genes play in biological processes, insights can be gained into the mechanisms underlying development, physiology, and disease. This knowledge is crucial for advancing our understanding of biology and for developing targeted interventions in areas such as medicine and agriculture.

In particularly, many of the both rare and common diseases are caused by genetic mutations. So, identifying the specific genes involved in development of such conditions is crucial for accurate diagnosis, prognosis, and treatment. For example, genetic testing can identify mutations associated with hereditary disorders like cystic fibrosis or Huntington's disease, enabling early detection and personalized treatment strategies that can have positive patient outcome.

In addition, identification of a particular gene can encode proteins that serve as targets for therapeutic interventions. Identifying the particular genes implicated in disease pathways can facilitate the discovery and development of new drugs. For instance, understanding the genetic basis of cancer can allows researcher to develop targeted therapies that specifically inhibit the activity of mutated genes or their products, minimizing side effects and improving patient outcomes.

Furthermore, identification and knowledge of specific genes can inform individuals about their risk of developing certain diseases or passing on genetic conditions to their offspring. So, gene identification can be basis for genetic counseling that helps individuals make informed decisions about family planning, screening, and preventive measures.

In agriculture culture, identifying genes associated with desirable traits such as yield, disease resistance, and nutritional content is crucial for crop improvement. This information enables breeders to develop new varieties through selective breeding or genetic engineering, contributing to food security and sustainable agriculture. Furthermore, identifying particular genes allows study of the evolutionary history of organisms and the genetic changes that has shaped biodiversity over time.

In the field of forensic science, identifying genes allows for the analysis of DNA evidence in criminal investigations, paternity testing, and disaster victim identification. DNA profiling, based on the identification of specific genetic markers, provides valuable information for solving crimes and resolving legal disputes.

The current method of gene identification primarily relies on high-throughput sequencing technologies coupled with computational analysis. This approach includes genome sequencing of the entire genome or specific regions of interest using high-throughput sequencing platforms such as Illumina or Pacific Biosciences. The sequencings are assembled into longer contiguous sequences, known as contigs, using specialized bioinformatics software. This step aims to reconstruct the original genome sequence from the short sequencing reads.

Once the genome is assembled, bioinformatics tools are employed to identify potential protein-coding genes within the genomic sequence. These tools search for characteristic features of genes, such as open reading frames (ORFs), promoter regions, and splice sites. After predicting genes, the identified sequences are annotated with information regarding their putative functions, such as protein domains, homologous sequences in other organisms, and functional annotations obtained from databases.

However, despite significant advancements, this method of gene identification has several limitations. The main limitation is that the genome assembly is a complex process, in particularly for large and repetitive genomes. As a consequence, genome assemblies may contain gaps, errors, or regions that are difficult to sequence and assemble accurately. This can lead to incomplete or fragmented gene annotations.

Another limitation is associated with automated gene prediction algorithms that may produce false-positive or false-negative results, leading to the mis-annotation of genes. Such errors can arise from inaccuracies in gene prediction models, misinterpretation of sequence features, or the presence of pseudogenes and non-coding regions.

Additionally, traditional gene prediction methods may struggle to accurately identify all splice variants, leading to incomplete annotations of gene structures and functions. Additionally, single-nucleotide polymorphisms (SNPs) and other genetic variations can further complicate gene identification and annotation efforts.

Another key limitation relates to functional annotation lacking or inaccuracies for many genes, especially in non-model organisms or those with poorly characterized genomes. Despite the advances in sequencing technologies, certain genomic regions, such as highly repetitive sequences or regions with extreme GC content, remain challenging to sequence accurately. These limitations can hinder gene identification efforts, particularly in complex or non-standard genomes.

Overall, there is need for methods of gene identification that is less complex and can overcome may challenges related to genome assembly, gene prediction, and functional annotation.

Accordingly, to overcome the disadvantages of the prior art, there is an urgent need for a technical solution that overcomes the above-stated limitations in the prior arts. The present invention provides a method for identifying a particular gene.

### SUMMARY OF THE INVENTION

The present disclosure solves all the above major limitations of method for identifying a particular gene. Further, the present disclosure ensures that the disclosed invention may fulfil following aspects:

An aspect of the present disclosure is to provide an effective and reliable method for identifying a particular gene.

Another aspect of the present disclosure is to provide a less complex method for identifying a particular gene.

Another aspect of the present disclosure is to provide a cost-effective method for identifying a particular gene.

Another aspect of the present disclosure is to provide a resource-efficient method for identifying a particular gene.

Another aspect of the present disclosure is to provide a cost-effective method for identifying a particular gene.

Another aspect of the present disclosure is to provide a method for identifying a particular gene that can aid in investigating the gene's allelic diversity and population-specific variations.

Another aspect of the present disclosure is to provide a method for identifying a particular gene that can reduce gaps and errors in gene identification.

Another aspect of the present disclosure is to provide a method for identifying a particular gene that can provide more accurate gene identification.

Another aspect of the present disclosure is to provide a method for identifying a particular gene that can facilitate studying gene orthologs, paralogs, and gene family relationships.

Yet another aspect of the present disclosure is to provide a method for identifying a particular gene that can aid in gaining insights into the biological significance, clinical relevance, and therapeutic potential of the gene of interest.

Yet another aspect of the present disclosure is to provide a method for identifying a particular gene that can reduce the risk of incomplete or fragmented gene annotations.

Yet another aspect of the present disclosure is to develop a method for identifying a particular gene that can reduce the risk of false-positive or false-negative results associated with automated gene prediction algorithms.

Yet another aspect of the present disclosure is to provide a method for identifying a particular gene that can aid reduce the risk of inaccuracies associated with gene prediction models.

Embodiments of the present invention relate to a method for identifying a particular gene in the genomic data. The method includes performing pre-processing on genomic data to remove any artifact or noise to ensure high quality data and the pre-processing on genomic data comprises normalization and transformation of data, filtering predictor variables and scaling, and discarding samples with missing values. The method also includes performing data analysis of the pre-processed genomic data to identify genes that are differentially expressed in specific conditions and the data analysis provides information on gene-variants analysis and gene expression. The method also includes performing data visualization to identify patterns or trends in the data being inapparent from the raw data and the data visualization facilitates exploring and presenting genomic data in a meaningful and interpretable manner. The method also includes validating the results of the data analysis to help confirm the identity of the identified gene and its association with specific conditions and the validating the results strengthen the validity and reliability of the data analysis.

In accordance with an embodiment of the present invention, the genomic data is obtained from a plurality of sources, including publicly available secure databases or data generated in-house.

In accordance with an embodiment of the present invention, the genomic data comprises gene expression data, deoxyribonucleic acid (DNA) sequence data, data on structure, function, evolution, mapping, or other types of genomic data.

In accordance with an embodiment of the present invention, the data analysis is performed using a variety of bioinformatics tools, such as statistical analysis tools, machine learning algorithms, or other data analysis methods.

In accordance with an embodiment of the present invention, the data visualization is performed using various visualization tools, such as heat maps, scatter plots, or other types of graphical representations.

In accordance with an embodiment of the present invention, the validation of the data is achieved using various experimental methods, such as qPCR, Western blotting, or other types of molecular biology techniques.

Another embodiment of the present invention, the method for identifying a particular gene from a tissue sample. The method includes obtaining a first sequence from the tissue sample and performing sequence inspection, the first sequence is associated with a particular gene. The method also includes obtaining a second sequence from the tissue sample and performing sequence inspection, the second sequence is associated with any other gene except the particular gene. The method also includes comparing the first sequence with a second sequence, the comparison determines whether the first sequence is different from the second sequence, and identifying the particular gene. The method also includes optionally obtaining a third sequence from the tissue sample and performing sequence inspection, the third sequence is associated with a different gene than the first and second sequences. The method also includes optionally comparing the third sequence with the first and second sequences, the comparison identifies the particular gene.

In accordance with an embodiment of the present invention, the tissue sample comprises a plurality of cells, and the first, second and third sequences are obtained by analyzing the cells using polymerase chain reaction (PCR), restriction fragment length polymorphism (RFLP), single nucleotide polymorphism (SNP), or any other suitable technique.

In accordance with an embodiment of the present invention, the sequence inspection is used to locate genes having distinctive features.

In accordance with an embodiment of the present invention, the first, second and third sequences are obtained from a secure database, including a public database, a proprietary database, or any other suitable database.

### BRIEF DESCRIPTION OF THE DRAWINGS

So that the manner in which the above-recited features of the present invention is understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

The invention herein will be better understood from the following description with reference to the drawings, in which:

FIG. 1 is a flowchart illustrating a method for identifying a particular gene in the genomic data, in accordance with one embodiment of the present invention; and

FIG. 2 is a flowchart illustrating a method for identifying a particular gene from a tissue sample, in accordance with one embodiment of the present invention.

It should be noted that the accompanying figure is intended to present illustrations of exemplary embodiments of the present disclosure. This figure is not intended to limit the scope of the present disclosure. It should also be noted that the accompanying figure is not necessarily drawn to scale.

### DETAILED DESCRIPTION OF THE INVENTION

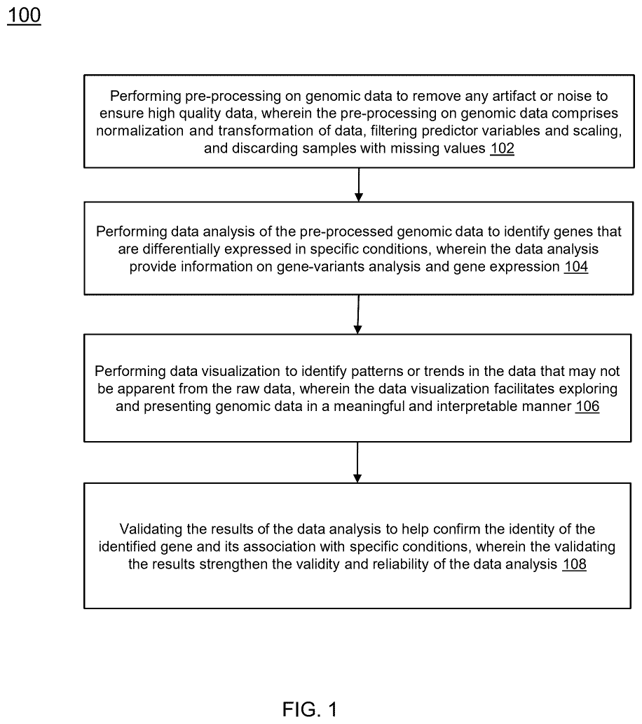

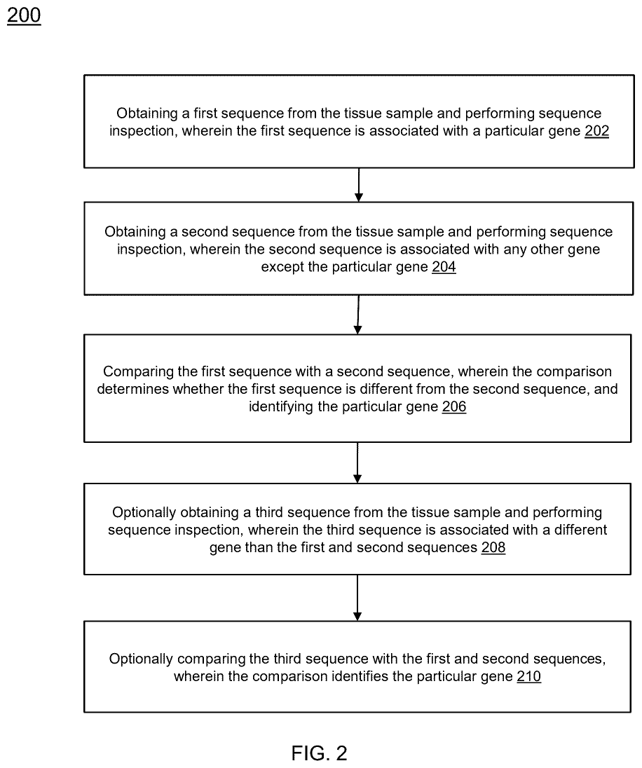

The principles of the present invention and their advantages are best understood by referring to FIGS. 1 and 2. In the following detailed description, numerous specific details are set forth in order to provide a thorough understanding of the embodiment of the invention as illustrative or exemplary embodiments of the invention, specific embodiments in which the invention may be practiced are described in sufficient detail to enable those skilled in the art to practice the disclosed embodiments. However, it will be obvious to a person skilled in the art that the embodiments of the invention may be practiced with or without these specific details. In other instances, well-known methods, procedures and components have not been described in detail so as not to unnecessarily obscure aspects of the embodiments of the invention.

The following detailed description is, therefore, not to be taken in a limiting sense, and the scope of the present invention is defined by the appended claims and equivalents thereof. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. References within the specification to “one embodiment,” “an embodiment,” “embodiments,” or “one or more embodiments” are intended to indicate that a particular feature, structure, or characteristic described in connection with the embodiment is included in at least one embodiment of the present invention.

Although the terms first, second, etc. may be used herein to describe various elements, these elements should not be limited by these terms. These terms are generally only used to distinguish one element from another and do not denote any order, ranking, quantity, or importance, but rather are used to distinguish one element from another. Further, the terms “a” and “an” herein do not denote a limitation of quantity, but rather denote the presence of at least one of the referenced items.

The conditional language used herein, such as, among others, “can,” “may,” “might,” “may,” “e.g.,” and the like, unless specifically stated otherwise, or otherwise understood within the context as used, is generally intended to convey that certain embodiments include, while other embodiments do not include, certain features, elements and/or steps.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

FIG. 1 is a flowchart illustrating a method 100 for identifying a particular gene in the genomic data, in accordance with one embodiment of the present invention. The method 100 comprises the following steps.

At 102, performing pre-processing on genomic data to remove any artifact or noise to ensure high quality data and the pre-processing on genomic data comprises normalization and transformation of data, filtering predictor variables and scaling, and discarding samples with missing values.

The genomic data may be obtained from various sources, including publicly available secure databases or data generated in-house.

The genomic data may include gene expression data, deoxyribonucleic acid (DNA) sequence data, data on structure, function, evolution, and mapping, or other types of genomic data.

In an embodiment of the present disclosure, tools used for pre-processing genomic data may be selected from the group of FastQC, Trimmomatic, Bowtie2, Burrows-Wheeler Aligner (BWA), Genome Analysis Toolkit (GATK), FreeBayes, Spliced Transcripts Alignment to a Reference (STAR), salmon, DESeq2, edgeR, Model-based Analysis of ChIP-seq (MACS2), Spatial Clustering for Identification of ChIP-Enriched Regions (SICER), ComBat, and such more.

In some embodiment, the pre-processing on genomic data includes performing quality control to remove low-quality sequences by checking read quality scores and filter out reads or sequences below a certain threshold.

In some embodiments, the pre-processing on genomic data includes use of alignment tools to convert into a format suitable for downstream analysis. The alignment tools may include Bowtie2, BWA, and/or HISAT2.

In some embodiment, the pre-processing on genomic data includes identifying genetic variations such as, SNPs, indels, structural variants) by comparing aligned sequences to the reference genome using tools like GATK, SAMtools, or FreeBayes for variant calling. In some embodiments, normalization read counts to account for differences in sequencing depth or library size, using methods like TPM (transcripts per million) or DESeq2 normalization.

In some embodiments, pre-processing of data includes performing differential gene expression analysis to identify genes that are differentially expressed between conditions. In some embodiments, pre-processing of data includes performing transformation, log transformation, and minimum-maximum scaling. In some embodiments, pre-processing of data includes selecting relevant gene features to improve model performance and integrate multi-dimensional data for a comprehensive analysis.

At 104, performing data analysis of the pre-processed genomic data to identify genes that are differentially expressed in specific conditions and the data analysis provide information on gene-variants analysis and gene expression.

The data analysis may be performed using a variety of bioinformatics tools, such as statistical analysis tools, machine learning algorithms, or other data analysis methods.

In an embodiment of the present disclosure, the data analysis may be performed on different types of genomic data, including gene expression data, DNA sequence data, or other types of genomic data.

In some embodiments, the data analysis may be Variant Analysis, including Single Nucleotide Polymorphisms (SNPs) Analysis, Structural Variants Analysis, and Variant Association Studies. In some embodiments, the data analysis may be Gene Expression Analysis including Differential Gene Expression Analysis, Isoform Analysis, and Co-expression Network Analysis.

In some embodiments, the data analysis may be Functional Genomics including Pathway and Functional Enrichment Analysis, Gene Set Enrichment Analysis (GSEA), and Regulatory Element Analysis. In some embodiments, the data analysis may be Structural Genomics including Chromatin Structure Analysis and Epigenetics Analysis.

In some embodiments, the data analysis may be Population Genomics including Population Genetics and Phylogenetics. In some embodiments, the data analysis may be Clinical Genomics that integrating genomic data with clinical information for personalized medicine, disease diagnosis, prognosis, and treatment selection and analyzes somatic mutations, gene expression profiles, and tumor heterogeneity to study cancer biology, drug response, and therapeutic targets. In some embodiments, the data analysis may be Systems Biology and Network Analysis including Systems Biology Modeling and Network Analysis.

In some embodiments, the tools for performing data analysis on genomic data may be selected from the group of Genome Analysis Toolkit (GATK), VarScan, DESeq2, edgeR, limma, Database for Annotation, Visualization, and Integrated Discovery (DAVID), Enrichr, Gene Set Enrichment Analysis (GSEA), Cytoscape, STRING, BioGRID, BreakDancer, CNVkit, SurvExpress, Kaplan-Meier Plotter, mixOmics, Seurat, and such others.

At 106, performing data visualization to identify patterns or trends in the data that may not be apparent from the raw data and the data visualization facilitates exploring and presenting genomic data in a meaningful and interpretable manner.

The data visualization may be performed using various visualization tools, such as heat maps, scatter plots, or other types of graphical representations.

In some embodiments, the data visualization may be Genome Browser Visualization using UCSC Genome Browser and/or Ensembl Genome Browser to allow visualizing genomic annotations, gene tracks, ChIP-seq peaks, and other features. In some embodiments, the data visualization may be performed using Alignment and Coverage Plots including Integrative Genomics Viewer (IGV) and/or Artemis for visualizing and displaying aligned sequencing reads, sequence features, and genomic annotations.

In some embodiments, the data visualization may be performed through Heatmaps and Clustering using Heatmap Visualization and Hierarchical Clustering for representing gene expression patterns, DNA methylation levels, and/or reveal structure and relationships within the data.

In some embodiments, the data visualization may be performed using Volcano Plots and Scatter Plots for visualizing differential gene expression or variant analysis results and comparing two variables to identify correlations, outliers, and patterns.

In some embodiments, the data visualization may be Network Visualization using Cytoscape and/or STRING, generation of Chromatin Interaction Maps using Hi-C Data Visualization Tools, Annotation and Pathway Visualization using Enrichment Plots and Pathway Maps, generation of Interactive Dashboards using Shiny (R) and/or Dash (Python), generation of Publication-Quality Figures using Adobe Illustrator, Inkscape.

At 108, validating the results of the data analysis to help confirm the identity of the identified gene and its association with specific conditions and the validating the results strengthen the validity and reliability of the data analysis.

The validation of the data may be achieved using various experimental methods, such as qPCR, Western blotting, or other types of molecular biology techniques.

In some embodiment, the validation of results is performed through experimental validation using PCR-based validation, sanger sequencing, and/or ChIP-qPCR. In some embodiment, the validation of results is performed through functional assays using cell culture experiments and/or CRISPR/CAS9 knockout or knockdown. In some embodiment, the validation of results is performed through comparative analysis by comparison with literature and cross-validation.

In some embodiment, the validation of results is performed through technical validation using replicate analysis and quality control checks. In some embodiment, the validation of results is performed through biological validation using functional enrichment analysis and survival analysis. In some embodiment, the validation of results is performed through collaborative validation through collaboration with domain experts and data sharing and reproducibility. In some embodiment, the validation of results is performed through statistical validation using statistical tests and false discovery rate (FDR) analysis.

In some embodiments, a combination of experimental, functional, comparative, and collaborative validation approaches may be employed, to strengthen the validity and reliability of genomic data analysis results and enhance the credibility of findings. In a preferred embodiment, the validation help confirm the identity of the identified gene and its association with specific conditions or specific medical conditions.

FIG. 2 is a flowchart illustrating a method 200 for identifying a particular gene from a tissue sample, in accordance with one embodiment of the present invention. The method 200 may comprise the following steps.

At 202, obtaining a first sequence from the tissue sample and performing sequence inspection, wherein the first sequence is associated with a particular gene.

At 204, obtaining a second sequence from the tissue sample and performing sequence inspection, wherein the second sequence is associated with any other gene except the particular gene.

At 206, comparing the first sequence with a second sequence, wherein the comparison determines whether the first sequence is different from the second sequence, and identifying the particular gene.

At 208, optionally obtaining a third sequence from the tissue sample and performing sequence inspection, wherein the third sequence is associated with a different gene than the first and second sequences.

At 210, optionally comparing the third sequence with the first and second sequences, wherein the comparison identifies the particular gene.

The tissue sample may comprise a plurality of cells, and the first, second and third sequences are obtained by analyzing the cells using polymerase chain reaction (PCR), restriction fragment length polymorphism (RFLP), single nucleotide polymorphism (SNP), or any other suitable technique.

The sequence inspection may be used to locate genes having distinctive features.

The first, second and third sequences may be obtained from a secure database, including a public database, a proprietary database, or any other suitable database.

In some embodiments, analysis of cells is performed to inspect gene sequences, analyze gene structure, identify functional elements, and investigate sequence variations.

In some embodiments, sequence inspection is performed using Basic Local Alignment Search Tool (BLAST) for searching search for similar sequences in nucleotide or protein databases and/pr helping to identify homologous genes or functional domains. In some embodiments, sequence inspection is performed using Ensembl Genome Browser allowing inspection of gene sequences, exons, introns, regulatory elements, and variations. In some embodiments, sequence inspection is performed using UCSC Genome Browser offering a genome visualization platform with rich annotations for genes, transcripts, regulatory elements, and genomic variants.

In some embodiments, sequence inspection is performed using Integrative Genomics Viewer (IGV) for visualizing and analyzing genomic data, including gene sequences, alignments, variants, and expression data.

In some embodiments, sequence inspection is performed using CLC Sequence Viewer for inspecting gene features and annotations. In some embodiments, sequence inspection is performed using Geneious Prime for exploring gene sequences, perform alignment-based comparisons, predict coding regions, and analyze sequence features. In some embodiments, sequence inspection is performed using Artemis Genome Browser to visualize gene structures, edit annotations, explore DNA sequences, identify motifs, and analyze genome organization.

In a best mode of operation, the method 200 for identifying a particular gene from a tissue sample includes obtaining a first sequence from the tissue sample and the first sequence is associated with a particular gene.

The method 200 also includes comparing the first sequence with a second sequence and the second sequence is associated with another gene. The method 200 also includes determining whether the first sequence is different from the second sequence, and thereby identifying the particular gene.

In conclusion, a method 100 for identifying a particular gene includes steps of data pre-processing, data analysis, data visualization and validation. The data analysis and data visualization aids in gaining insights into genomic data, identifying patterns, correlations, and biological significance, and communicating findings effectively. The validation of the results of genomic data analysis ensures the reliability, reproducibility, and biological relevance of findings.

The disclosed invention is less complex, more cost-effective, and more resource-efficient than the traditional method of identifying a gene. The disclosed invention may find application in identification of the biological function of a particular gene, such as its role in cellular processes, pathways, or disease mechanisms. The disclosed invention may aid in determining gene interactions with other genes, proteins, or regulatory elements in the genome.

The disclosed invention may aid in identifying the gene as a diagnostic or prognostic marker for a particular disease or condition and investigating the gene's expression patterns, mutations, or epigenetic modifications as potential biomarkers. The disclosed invention may also help in identifying the gene as a potential therapeutic target for drug development or precision medicine approaches and characterize the gene's involvement in disease pathways and assess its effectiveness.

The disclosed invention may also facilitate identifying genetic variants within the gene and assess their association with phenotypic traits, diseases, or drug responses. The disclosed invention may also reduce the risk of incomplete or fragmented gene annotations. false-positive or false-negative results associated with conventional gene prediction algorithms. And inaccuracies associated with conventional gene prediction models.

In a case that no conflict occurs, the embodiments in the present disclosure and the features in the embodiments may be mutually combined. The foregoing descriptions are merely specific implementations of the present disclosure, but are not intended to limit the protection scope of the present disclosure. Any variation or replacement readily figured out by a person skilled in the art within the technical scope disclosed in the present disclosure shall fall within the protection scope of the present disclosure. Therefore, the protection scope of the present disclosure shall be subject to the protection scope of the claims.

The foregoing descriptions of specific embodiments of the present technology have been presented for purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology.

What is claimed for:

1. A method for identifying a particular gene in the genomic data, the method comprising:
performing pre-processing on genomic data to remove any artifact or noise to ensure high quality data;
performing data analysis of the pre-processed genomic data to identify genes that are differentially expressed in specific conditions;
performing data visualization to identify patterns or trends in the data that being inapparent from the raw data, wherein the data visualization facilitates exploring and presenting genomic data in a meaningful and interpretable manner; and
validating the results of the data analysis to help confirm the identity of the identified gene and its association with specific conditions, wherein the validating the results strengthen the validity and reliability of the data analysis.

2. The method of claim 1, wherein the pre-processing on the genomic data comprises normalization and transformation of data, filtering predictor variables and scaling, and discarding samples with missing values.

3. The method of claim 1, wherein the data analysis provide information on gene-variants analysis and gene expression.

4. The method of claim 1, wherein the genomic data is obtained from a plurality of sources, including publicly available secure databases or data generated in-house.

5. The method of claim 1, wherein the genomic data comprises gene expression data, deoxyribonucleic acid (DNA) sequence data, data on structure, function, evolution, mapping, or other types of genomic data.

6. The method of claim 1, wherein the data analysis is performed using a variety of bioinformatics tools comprising statistical analysis tools, machine learning algorithms, or other data analysis tools.

7. The method of claim 1, wherein the data visualization is performed using various visualization tools, such as heat maps, scatter plots, or other types of graphical representations.

8. The method of claim 1, wherein the validation of the data is achieved using various experimental techniques comprising qPCR, Western blotting, or other types of molecular biology techniques.

9. A method for identifying a particular gene from a tissue sample, the method comprising:
obtaining a first sequence from the tissue sample and performing sequence inspection, wherein the first sequence is associated with a particular gene;
obtaining a second sequence from the tissue sample and performing sequence inspection, wherein the second sequence is associated with any other gene except the particular gene;
comparing the first sequence with a second sequence, wherein the comparison determines whether the first sequence is different from the second sequence, and identifying the particular gene;
optionally obtaining a third sequence from the tissue sample and performing sequence inspection, wherein the third sequence is associated with a different gene than the first and second sequences; and
optionally comparing the third sequence with the first and second sequences, wherein the comparison identifies the particular gene.

10. The method of claim 9, wherein the tissue sample comprises a plurality of cells, and the first, second and third sequences are obtained by analyzing the cells using polymerase chain reaction (PCR), restriction fragment length polymorphism (RFLP), single nucleotide polymorphism (SNP), or any other suitable technique.

11. The method of claim 9, wherein the sequence inspection is used to locate genes having distinctive features.

12. The method of claim 9, wherein the first, second and third sequences are obtained from a secure database, including a public database, a proprietary database, or any other suitable database.
